(19) United States
(12) Patent Application Publication
(10) Pub. No.: US 2021/0378918 A1
(43) Pub. Date: Dec. 9, 2021

(54) COMPOSITIONS AND COATINGS FOR SOFT TISSUE ATTACHMENT

(71) Applicant: REGENTS OF THE UNIVERSITY OF MINNESOTA, Minneapolis, MN (US)

(72) Inventors: Conrado Aparicio, Minneapolis, MN (US); Nicholas G. Fischer, Minneapolis, MN (US)

(21) Appl. No.: 17/339,243

(22) Filed: Jun. 4, 2021

Related U.S. Application Data
(60) Provisional application No. 63/034,752, filed on Jun. 4, 2020.

(51) Int. Cl. A61K 6/30; A61L 29/08; A61L 27/34; C08F 222/10

(52) U.S. Cl. CPC A61K 6/30; A61L 29/085; A61L 27/34; C08F 2810/20; C08F 222/104; A61L 2420/02; C08F 222/103

(57) ABSTRACT

An ultraviolet photopolymerizable composition for soft tissue attachment includes trimethylolpropane tris(3-mercaptopropionate) (TMTMP), a photoinitiator, a solvent, and one or more of trimethylolpropane trimethacrylate (TMPTMA), pentaerythritol triacrylate (PETA), dopamine methacrylamide (DMA), and n-phenethylmethacrylamide (PEMAD). Coating a substrate with a polymeric coating includes contacting a substrate with the ultraviolet photopolymerizable and irradiating the composition with ultraviolet radiation to yield a polymeric coating on the substrate. Treating a substrate includes contacting the substrate with the composition and irradiating the composition with ultraviolet radiation to yield a polymeric coating on the substrate.

### CROSS-REFERENCE TO RELATED APPLICATION

This application claims the benefit of U.S. patent application Ser. No. 63/034,752 entitled “COMPOSITIONS AND COATINGS FOR SOFT TISSUE ATTACHMENT” and filed on Jun. 4, 2020, which is incorporated herein by reference in its entirety.

### STATEMENT OF GOVERNMENT INTEREST

This invention was made with government support under 5R01DE026117 and F30DE029105 awarded by NIH-NIDCR. The government has certain rights in this invention.

### TECHNICAL FIELD

This invention relates to coatings and compositions for soft tissue attachment.

### BACKGROUND

An estimated 800 million resin composite, 100 million amalgam, and millions of glass ionomer cement restorations are placed each year worldwide and are together one of the most prevalent medical interventions in the human body. Elderly individuals (65 years plus) are eight times more prone to restorations on the lower third of the tooth, the so-called Class V restoration, than middle-aged individuals. Concurrently, the U.S. population proportion of elderly individuals is rising 62% from 2000 to 2030. Longer tooth retention, deeper probing pocket depths, increased root caries, and expansion of nursing homes over the past 20 years has fueled the increase in frequency of these restorations amongst elderly individuals. However, modern restorative materials are not suited to meet this challenge. Restorations for these caries fail sooner and at a higher rate than any other class of restorations. Materials developed to combat the high failure rate of these percutaneous restorations have typically focused on mechanical properties of the filling material or adhesive properties of the adhesives used.

In parallel, a wide variety of other percutaneous devices (any biomedical device that breaks the skin or other mucosa to penetrate into tissues and stay temporarily or permanently placed with an exit point from the body forming a skin-device interface) show high failure rates. For example, indwelling catheters are responsible for 80,000 infections, 20,000 deaths, and an associated cost as high as US$2.3 billion annually. Simultaneously, one million dental implants worldwide fail per year and have a functional lifespan of only five to 11 years. Percutaneous osseointegrated prosthesis for amputees, such as those caused by dysvascular disease and trauma, are associated with 40% of patients suffering skin break-down and infection in up to 77% of patients.

### SUMMARY

This disclosure describes compositions for soft tissue attachment, such as polymerizable compositions for coating restorations where soft tissue attachment is desirable, such as Class IIs and Class Vs oral restorations or root caries. In one example, “soft tissue” generally refers to skin and oral mucosa. These polymerizable compositions are also suitable for coatings directly the tooth to promote soft tissue attachment, for catheters (dialysis, ventricular assisted devices) or osseointegrated, percutaneous devices such as orthopedic limb prostheses, dental implants, or bone-anchored hearing aids.

Compositions described herein overcome causes of Class V and other percutaneous device failure by regenerating the junctional epithelium (JE) or soft tissue (e.g., hemidesmosome formation by keratinocytes) on the restoration or other device surface. This hemidesmosomes formation leads to extended lifespans and reduced device failure as this is the native role of JE on the long-lasting tooth surface. Further, the clinical procedure for application typically includes a coating step but does not require alteration to existing materials.

A first general aspect includes an ultraviolet photopolymerizable composition for soft tissue attachment. The composition includes trimethylolpropane tris(3-mercaptopropionate) (TMTMP), a photoinitiator, a solvent, and one or more of trimethylolpropane trimethacrylate (TMPTMA), pentaerythritol triacrylate (PETA), dopamine methacrylamide (DMA), and n-phenethylmethacrylamide (PEMAD).

Implementations of the first general aspect may include one or more of the following features.

In some implementations, the photoinitiator includes dimethylol propionic acid (DMPA). The composition typically includes 0.5 w/v to 5 w/v or 0.5 w/v to 2 w/v of the photoinitiator.

In some implementations, the solvent includes one or more of water, methanol, ethanol, acetone, tetrahydrofuran (THF), ethyl ether, and dichloromethane (DCM). The composition typically includes 5% v/v to 40% v/v of the solvent.

In some implementations, the composition includes TMPTMA and PETA. A molar ratio of TMPTMA and PETA to TMTMP can be in a range of 1:0 to 4:0. In some cases, the composition includes up to 50 mM DMA, up to 50 mM PEMAD, or both.

In some implementations, the composition promotes keratinocyte proliferation, adhesion, hemidesmosome formation, or any combination thereof.

In a second general aspect, coating a substrate with a polymeric coating includes contacting a substrate with the photopolymerizable composition of the first general aspect and irradiating the photopolymerizable composition with ultraviolet radiation to yield a polymeric coating on the substrate.

Implementations of the second general aspect may include one or more of the following features.

In some implementations, the ultraviolet radiation has a wavelength of 365 nm or approximately 365 nm. Irradiating can include irradiating at an intensity of at least 2 mWcm⁻² for at least 20 seconds. Some implementations of the second general aspect further include contacting the polymeric coating with soft tissue.

A third general aspect includes a coated substrate formed by the method of the second general aspect.

Implementations of the third general aspect may include one or more of the following features.

In some implementations, the substrate includes a device configured to be at least partially inserted in a mammalian body or in contact with soft tissue (e.g., skin or oral mucosa) in a mammalian body. Suitable substrates include dental restorations, a catheter, or osseointegrated percutaneous devices. Examples of osseointegrated percutaneous device include orthopedic limb prostheses, dental implants, and bone-anchored hearing aids. Examples of catheters include dialysis catheters and ventricular assisted devices. The dental restorations can be at least partially in a root of a tooth.

In a fourth general aspect, treating a substrate includes contacting the substrate with the composition of the first general aspect, and irradiating the composition with ultraviolet radiation to yield a polymeric coating on the substrate. The substrate can include a dental restoration, a catheter, or an osseointegrated percutaneous device, and the polymeric coating promotes soft tissue attachment to the substrate.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 depicts a Class V restoration with a composition for soft tissue attachment.

FIG. 2 depicts chemical components in compositions for soft tissue attachment.

FIG. 3 shows adherence of a composition for soft tissue attachment to an applicator brush.

FIG. 4 shows degree of conversion (chemical polymerization and polymer crosslinking) of compositions for soft tissue attachment.

FIG. 5 shows Vickers microhardness (HV) of polymerizable compositions for soft tissue attachment.

FIGS. 6A and 6B show simulated oral degradation at pH=8.5 (gingival crevicular fluid pH) and pH=7.4 (standard in vitro degradation assay pH), respectively, as assessed by mass loss (% of original mass).

FIGS. 7A and 7B show human oral keratinocytes proliferation (number of cells per field of view (FOV)) after 1 and 3 days of culture, respectively.

FIGS. 8A and 8B show human oral keratinocytes proliferation (CCK8 metabolic activity; optical density (OD)) after 1 and 3 days of culture, respectively.

FIGS. 9A and 9B show human oral keratinocytes surface area/spreading (SA) after 1 and 3 days of culture, respectively, as fold-change compared to glass slides.

FIGS. 10A and 10B show human oral keratinocyte hemidesmosome formation (β4 integrin) immunofluorescence intensity after 1 and 3 days of culture, respectively.

FIGS. 11A and 11B show human oral keratinocyte hemidesmosome formation (Collagen XVII) immunofluorescence intensity after 1 and 3 days of culture, respectively.

FIGS. 12A and 12B show human oral keratinocyte hemidesmosome formation (plectin) immunofluorescence intensity after 1 and 3 days of culture, respectively.

FIG. 13 shows keratinocyte (derived from rat tail epidermis) proliferation (CCK8 metabolic activity) of example compositions compared to collagen I coated glass slides, expressed as fold change of collagen coated surfaces.

FIGS. 14A and 14B show human gingival fibroblast proliferation (number of cells per field of view (FOV)) after 1 and 3 days of culture, respectively.

FIGS. 15A and 15B show human gingival fibroblast proliferation (CCK8 metabolic activity) after 1 and 3 days of culture, respectively.

FIG. 16 shows human oral keratinocyte cytotoxicity (via LDH release) after 1 day of culture.

FIG. 17 shows human oral keratinocyte PI3K (phosphoinositide 3-kinase) activity (downstream of hemidesmosome formation) via a modified ELISA.

FIGS. 18A and 18B show human oral keratinocyte formation proliferation marker (Ki-67; percent of total cells positive for the marker) immunofluorescence expression after 1 and 3 days of culture, respectively.

FIG. 19 shows human oral keratinocyte mechanical adhesion (percent adhered compared to non-centrifuged) to compositions after centrifugation at varying RCF (g) values.

FIG. 20 shows human oral keratinocyte SLPI (secretory leukocyte peptidase inhibitor) expression at homeostasis (control) and after exposure to IL-1α (interleukin 1α; stimulated).

FIG. 21 shows human oral keratinocyte LL-37 expression at homeostasis and after exposure to LPS (lipopolysaccharide; stimulated).

FIG. 22 shows failure load during scratch test for mechanical attachment to substrate after a thin layer of composition for soft tissue attachment is placed on a commercial Class V filling material (Z250; 3M Oral Care Solutions).

FIG. 23 shows rheology shear rate sweep of compositions for soft tissue attachment.

FIG. 24 shows swelling ratio (comparison of weight before and after immersion in phosphate buffered saline) of compositions for soft tissue attachment.

FIG. 25 shows human oral keratinocyte proliferation (metabolic activity; 1 day) on TMPTMA and PETA compositions after hydrolytic degradation for 1 week.

FIGS. 26A and 26B show human oral keratinocytes proliferation (CCK8 metabolic activity) after 1 and 3 days of culture, respectively, after fouling of compositions with bovine serum albumin (BSA).

FIGS. 27A and 27B show human oral keratinocytes proliferation (number of cells per field of view (FOV)) after 1 and 3 days of culture, respectively, after fouling of compositions with BSA.

FIGS. 28A and 28B show human oral keratinocyte hemidesmosome formation (Collagen XVII) immunofluorescence intensity after 1 and 3 days of culture, respectively, after fouling of compositions with BSA.

FIG. 29 shows sessile drop water contact angle of compositions and representative images, respectively.

FIG. 30 shows surface free energy (SFE; γ^{s}) of compositions determined with contact angles. Values are reported as γ^{d}, γₚ, and y^{h} components of SFE arising from the dispersion force, the polar (permanent and induced) force, and the hydrogen-bonding force, respectively.

FIG. 31 shows contact angles of various solutions of water with similar ionic strengths at pH=2 to pH=12 on all compositions.

FIG. 32 shows surface roughness (Rₐ) of compositions determined with a white-light confocal laser microscope.

FIGS. 33A and 33B show scanning electron micrographs of a cross section with a TMPTMA composition adhered to a commercially available restorative material before and after the equivalent of 9.25 years of toothbrushing, respectively.

FIG. 34 shows protein adsorption (via bicinchoninic acid assay (BCA) assay) from keratinocytes and fibroblast media on compositions.

FIGS. 35A-35D show water contact angle and human oral keratinocyte proliferation ((CCK8) metabolic activity; optical density (OD)), hemidesmosome formation (Collagen XVII), and hemidesmosomes formation (β4 integrin) after 1 day of culture, respectively, on DMA varied concentration in TMPTMA.

FIG. 36 shows surface zeta potential (SZP) of compositions for soft tissue attachment.

FIG. 37 shows integrin function-perturbing blocking studies to show human oral keratinocyte reliance on integrins for adhesion to compositions for soft tissue attachment.

### DETAILED DESCRIPTION

Compositions described in this disclosure can be applied inside or outside of the mouth to surfaces and polymerized to yield a polymeric coating that promotes soft tissue attachment to dental restorations and percutaneous devices such as dental implants, orthopedic implants, and dialysis catheters. The polymeric coating enhances tissue attachment and reduces overall failure rates due at least in part to infection. Although these compositions and coatings are generally applicable to attachment of various types of soft tissue (e.g., skin) to various surfaces and devices, examples described herein are related to soft tissue attachment to a restoration on the root of a tooth.

Placement of restorations in the lower third of a tooth (i.e., the root) can break the existing seal between the gingiva and the tooth at the junctional epithelium (JE) and traumatize soft tissue. JE mediates attachment of gingiva to teeth and prevents subgingival plaque or bacteria that could lead to recurrent caries (infection), inflammation, and bone loss. The JE is the first line of defense the interface between the gingiva and tooth has against harsh conditions in the oral cavity. However, the JE typically does not reform on existing restorative materials. This can lead to subgingival plaque accumulation, further apical migration, more exposed root and restoration surface, and contribute to restoration failure. As disclosed herein, a functional JE achieved through formation of hemidesmosomes on Class V restorations, as an exemplar percutaneous device, can naturally prevent subgingival plaque or bacteria and lead to longer restoration lifespan by exploiting the protective functions of the native JE tissue.

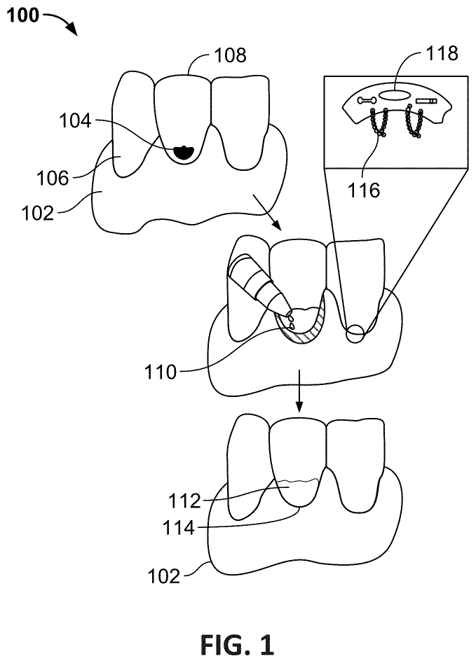

FIG. 1 depicts process 100 for applying a polymerizable composition for soft tissue 102 attachment to a restoration surface 104 on the root 106 of a tooth 108. The polymerizable composition 110 is photopolymerizable, and is cured (e.g., by irradiation with ultraviolet (UV) light) to yield a polymeric coating 112 on the restoration surface 104. The soft tissue 102 of the JE 114 attaches to the polymeric coating 112 through hemidesmosome formation, thereby restoring soft tissue attachment to the tooth at the restoration surface 104. Hemidesmosomes 116 are small structures found in keratinocytes 118 of JE 114 that attach to the extracellular matrix. This soft tissue attachment helps extend the functional lifetime of the restoration, for example, by guiding keratinocyte hemidesmosome formation in a manner similar to that of native JE. In a similar fashion, catheters (dialysis, ventricular assisted devices) and osseointegrated, percutaneous devices such as orthopedic limb prostheses, dental implants, and bone-anchored hearing aids destroy surrounding soft tissues, thus increasing vulnerability to infection. These compromised soft tissues would benefit from keratinocyte hemidesmosome formation.

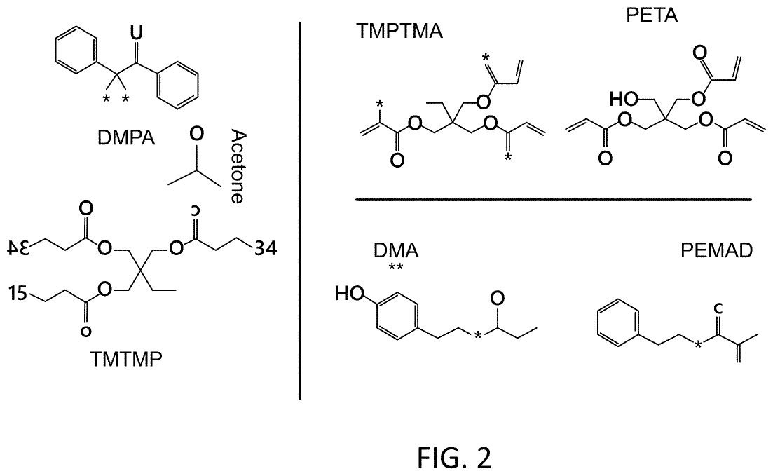

Compositions for soft tissue attachment include a photocrosslinking system and one or more additional monomers. The photocrosslinking system includes trimethylolpropane tris(3-mercaptopropionate) (TMTMP), a photoinitiator, and a solvent. One example of a suitable photoinitiator is 2,2-dimethoxy-2-phenylacetophenone (DMPA). The photocrosslinking system is a composition that polymerizes upon exposure to ultraviolet (UV) radiation (e.g., 365 nm). Suitable solvents include water, methanol, ethanol, acetone, tetrahydrofuran (THF), ethyl ether, dichloromethane (DCM), and combinations thereof. The additional monomers include trimethylolpropane trimethacrylate (TMPTMA), pentaerythritol triacrylate (PETA), 2,2-dopamine methacrylamide (DMA), and n-phenethylmethacrylamide (PEMAD). FIG. 2 shows chemical structures for DMPA, TMTMP, TMPTMA, PETA, DMA, and PEMAD. During synthesis, components of the photocrosslinking system and one or more of the additional monomers are combined to yield a polymerizable composition.

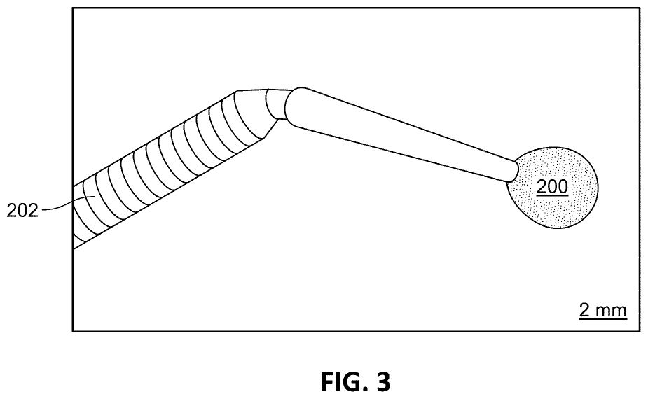

The polymerizable composition is dispensed or applied (e.g., from a bottle or a syringe), as depicted in FIG. 1, onto a surface or an applicator brush. FIG. 3 shows a polymerizable composition 200 for soft tissue attachment on an applicator brush 102. The polymerizable (TMPTMA-based) composition can be dyed with methylene blue. The composition on the applicator brush can be applied to a surface (e.g., the surface of a Class V restoration) and then polymerized (e.g., photopolymerized with UV) to yield a polymeric coating on the surface that promotes attachment of soft tissue to the device surface via upregulation of keratinocyte hemidesmosomes.

### EXAMPLES

Material Synthesis and Composition. TMPTMA (trimethylolpropane trimethacrylate; 246840, Millipore Sigma) and PETA (pentaerythritol triacrylate; 246794, Millipore Sigma) were mixed with a crosslinking reagent, TMTMP (trimethylolpropane tris(3-mercaptopropionate); 381489, Millipore Sigma), at a molar ratio of 1.4:1 (acrylate:thiol) and 1% w/v of photoinitiator DMPA (2,2-dimethoxy-2-phenylacetophenone; 196118, Millipore Sigma) without further purification. Acetone was added at 10% (v/v). In some cases, acetone was laden with either custom-synthesized PEMAD (n-phenethylmethacrylamide; Polymer Source Inc.) or DMA (dopamine methacrylamide; Polymer Source Inc.) for a final concentration of 4.2 mM. Example compositions were mixed on a carousel rotatory mixer overnight. Photopolymerization was achieved with approximately 365 nm UV-irradiation at an intensity of approximately 2 mW cm⁻² for at least one minute (i.e., to completion). Controls including a commercially available pit and fissure sealant (Helioseal, Vioclar Vivadent) and resin modified class ionomer cement (Ketac Nano, 3M Oral Care) were obtained for comparison as clinically used percutaneous materials. All compositions and controls were photopolymerized with an Elipar DeepCure-S (3M Oral Care).

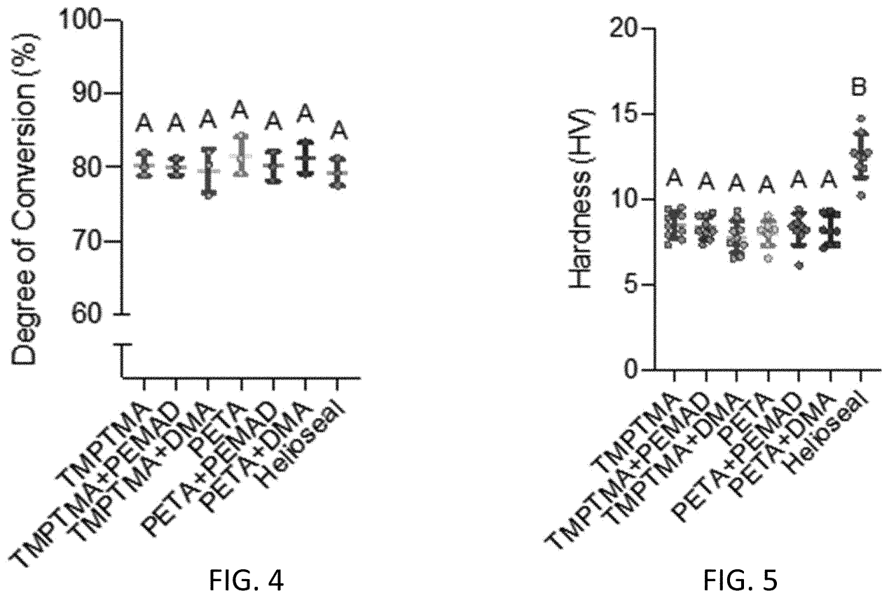

Example 1. TMPTMA and PETA were crosslinked with TMTMP and DMPA with the addition of small v/v % of DMA or PEMAD for similar degree of conversions to form a thin film or coating on underlying substrates. ATR-FTIR spectroscopy was performed on a Nicolet iS50 FTIR (Thermo Fisher) at a resolution of 2 cm⁻¹ from 400-4000 cm⁻¹. One drop of the monomeric or photopolymerized example compositions were placed on the diamond plate of an attenuated total reflectance (ATR) accessory. Degree of conversion (DC) for each composition was determined by comparing the ratio between the peak height of the C—H vinyl group (810 cm⁻¹) and C—O alcohol group absorptions for monomeric and photopolymerized example compositions. DC was calculated following conventional methods for Helioseal. See, e.g., Gonalves, et al., Influence of BisGMA, TEGDMA, and BisEMA Contents on Viscosity, Conversion, and Flexural Strength of Experimental Resins and Composites. Eur. J. Oral Sci. 2009, 117 (4), 442-446 DOI: 10.1111/j.1600-0722.2009.00636.x. FIG. 4 shows degree of conversion (chemical polymerization and polymer crosslinking) of the example compositions. A commercial pit and fissure sealant (Helioseal, Ivoclar Vivadent) was tested as a control. Statistical analysis: One-way ANOVA; Tukey post-hoc; letters with different symbols were significantly different (p<0.05). No differences were seen between the example compositions and the control, demonstrating that the example compositions have sufficient crosslinking for clinical utilization.

Example 2. The Vickers microhardness (VH) of polymerized example compositions was determined with a microindentation hardness tester (Micromet 5104, Buehler) with a 50 g load applied for 20 s. FIG. 5 shows Vickers microhardness (HV) of polymerized example compositions. A commercial pit and fissure sealant (Helioseal) was tested as a control. Statistical analysis: One-way ANOVA; Tukey post-hoc; letters with different symbols were significantly different (p<0.05). No differences in hardness were observed between the example compositions and the control, suggesting that differences in cellular response are not due to mechanical properties. Hardness values for the example compositions were lower than Helioseal, which contains Bis-GMA (bisphenol A-glycidyl methacrylate), a relatively rigid monomer.

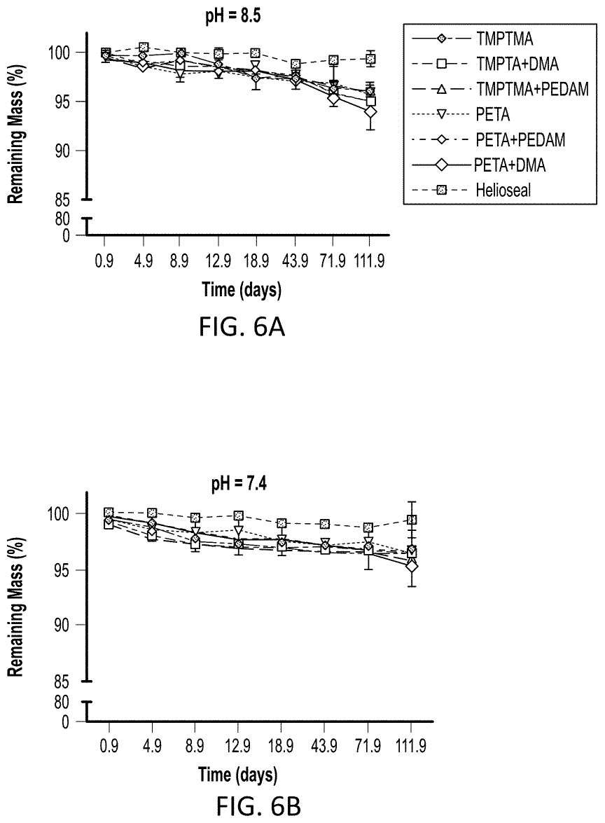

Example 3. The water-mediated degradation profile (weight loss) of all crosslinked example compositions is similar. Degradation of example compositions was assessed by mass loss after incubation at pH=7.4 [phosphate buffered saline, (PBS]) and pH=8.5 [tris buffered saline, (TBS)] at 37° C. for various lengths of time up to 453 days. Samples were periodically removed, dessicated 72 hours, and then massed (resolution of 0.1 mg; Sartorius Entris 64-12). FIGS. 6A and 6B show simulated oral degradation at pH=8.5 (gingival crevicular fluid pH) and pH=7.4 (standard in vitro degradation assay pH), respectively, as assessed by mass loss (% of original mass). A commercial pit and fissure sealant (Helioseal) was tested as a control. No differences were observed in mass loss between any of the example compositions. Mass loss for all example compositions was below 10% in +1.2 years. Helioseal, composed of di-methacrylates compared to tri-meth/acrylates of the example compositions, where meth/acrylates are water-labile, degrades at a slower rate than the example compositions.

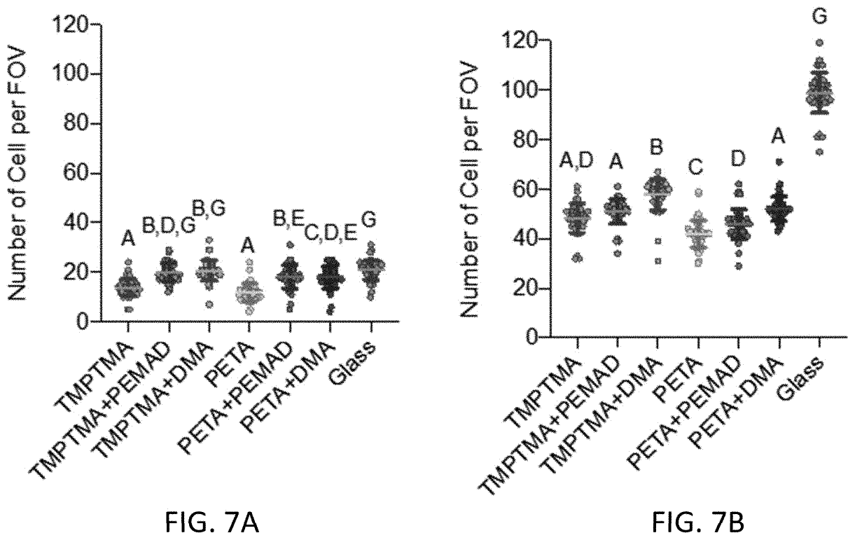

Example 4. Incorporation of DMA and PEMAD into both TMPTMA and PETA compositions increases keratinocyte proliferation as measured by metabolic activity, number of cells, and percentage of cells expressing Ki-67 cell proliferation marker. This is not due to difference in cytotoxicity. Immortalized human TERT-2/OKF-6 (BWH Cell Culture and Microscopy Core, Boston, MA, USA) oral keratinocytes, from non-neoplastic tissue from of the floor of the mouth were cultured in defined keratinocyte serum-free media (17005042, Gibco) with 1% penicillin/streptomycin (15140148, Gibco) under standard conditions. See Dickson et al., Human Keratinocytes That Express HTERT and Also Bypass a P16INK4a-Enforced Mechanism That Limits Life Span Become Immortal yet Retain Normal Growth and Differentiation Characteristics. Mol. Cell. Biol. 2000, 20 (4), 1436-1447 DOI: 10.1128/MCB.20.4.1436-1447.2000. Cells were seeded at 5,000 cells per well in a 48 wellplate for all experiments. Cell-laden disks were fixed, permeabilized, and blocked at each timepoint. Rhodamine-conjugated phalloidin (R415, Thermo-Fisher) was diluted in 5% BSA for 10 minutes at room temperature following manufacturer's instructions. Standard immunofluorescence was then performed to quantify the number of cells per field of view. Visualization was performed on a Leica DM6 B upright fluorescent microscope at×10 (0.32 PH1 at 1296×966 pixels) and analyzed in ImageJ (NIH). FIGS. 7A and 7B show oral keratinocytes proliferation (number of cells per field of view (FOV)) after 1 and 3 days of culture, respectively. Glass slides were tested as a control group for the experiment. Statistical analysis: One-way ANOVA; Tukey post-hoc; letters with different symbols were significantly different (p<0.05). Inclusion of DMA or PEMAD into TMPTMA or PETA increased the number of cells per field of view at both timepoints (1 and 3 days). TMPTMA-based example compositions tended higher than PETA-based example compositions. All example composition values were lower than that of the glass positive control.

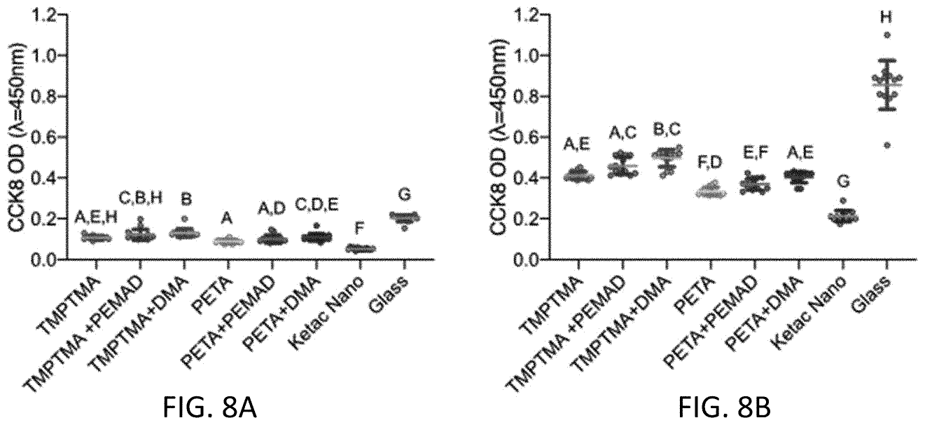

Metabolic activity was determined with a Cell Counting Kit 8 (CCK8, Dojindo) at one and three days of culture. Disks were incubated in CCK8 solution for three hours following manufacturer's instructions. Absorbance (optical density) was read using a microplate reader (Synergy HT, Biotek) at 450 nm. FIGS. 8A and 8B show human oral keratinocytes proliferation (CCK8 metabolic activity; optical density (OD)) after 1 and 3 days of culture, respectively. Glass slides and a commercially available restorative material (Ketac Nano) were tested as a control group for the experiment. Statistical analysis: One-way ANOVA; Tukey post-hoc; letters with different symbols were significantly different (p<0.05). Inclusion of DMA or PEMAD into TMPTMA or PETA increased metabolic activity at both timepoints (1 and 3 days). TMPTMA-based example compositions tended higher than PETA-based example compositions. All example composition values were lower than that of the glass positive control but higher than the existing, commercially available material Ketac Nano. Thus, existing monomer technologies are typically cytotoxic and therefore do not yield hemidesmosome formation.

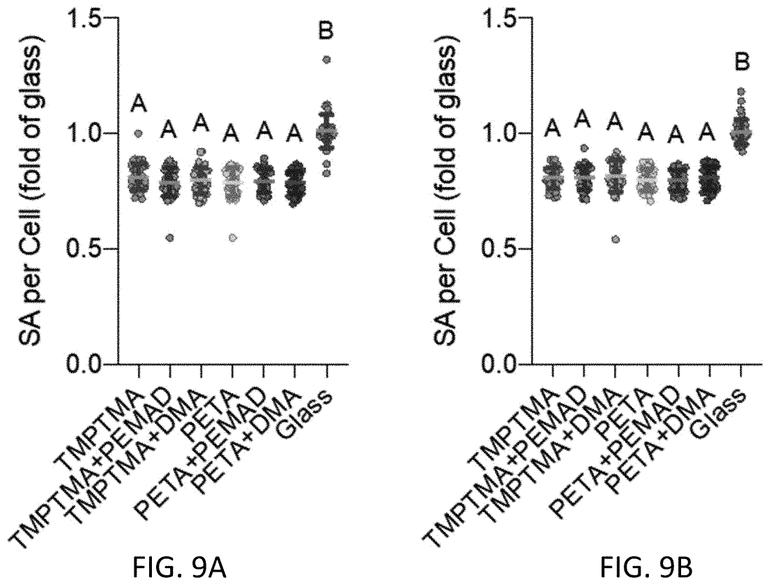

Example 5. Incorporation of DMA and PEMAD into both TMPTMA and PETA example compositions does not affect keratinocyte size (surface area). The previously performed immunofluorescence against rhodamine-conjugated phalloidin was quantified in ImageJ. FIGS. 9A and 9B show human oral keratinocytes surface area/spreading (SA) after 1 and 3 days of culture, respectively, as fold-change compared to glass slides. Glass slides were tested as a control group for the experiment. Statistical analysis: One-way ANOVA; Tukey post-hoc; letters with different symbols were significantly different (p<0.05). Inclusion of DMA or PEMAD into TMPTMA or PETA did not affect cell spreading. Cell spreading was higher in all cases on the positive control glass; this is likely related to mechanical stimuli.

Example 6. Incorporation of DMA and PEMAD into both TMPTMA and PETA example compositions increases keratinocyte hemidesmosome formation as measured by three hemidesmosome markers: integrin (34, collagen XVII, and plectin. Immunofluorescence staining was performed to semi-quantitively measure hemidesmosome. Cells were seeded at 5,000 cells per 48 wellplate well for all experiments. Cell-laden disks were fixed, permeabilized, and blocked at each timepoint. Glass cover slips were used a positive controls for all biological experiments. Next, example compositions were incubated in appropriate antibody solutions (see table) at room temperature for one hour. Secondary antibodies were applied at room temperature for one hour. Samples were then visualized with immunofluorescent microscopy as described.

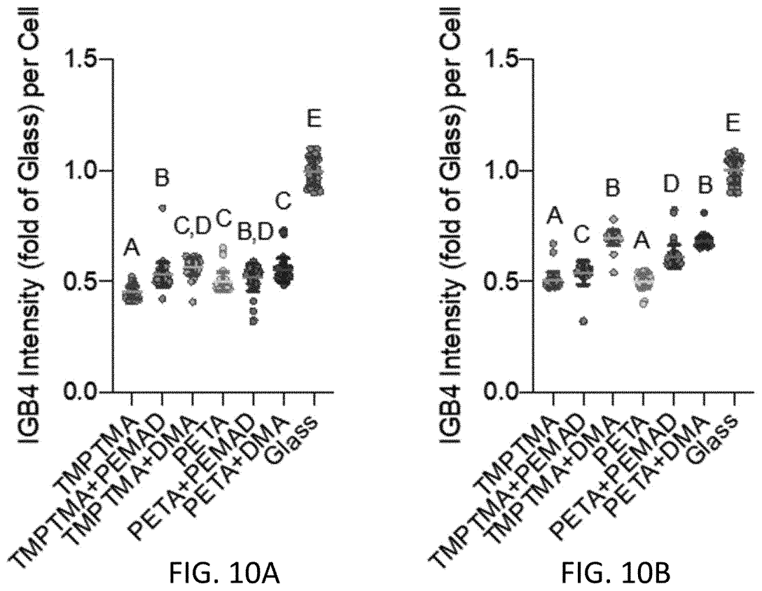

FIGS. 10A and 10B show human oral keratinocyte hemidesmosome formation (IGB4; β4 integrin) immunofluorescence intensity after 1 and 3 days of culture, respectively. Glass slides were tested as a control group for the experiment. Statistical analysis: One-way ANOVA; Tukey post-hoc; letters with different symbols were significantly different (p<0.05). Inclusion of DMA or PEMAD into TMPTMA or PETA increased hemidesmosome formation at both timepoints (1 and 3 days). TMPTMA-based example compositions tended higher than PETA-based example compositions. All example composition values were lower than that of the glass positive control.

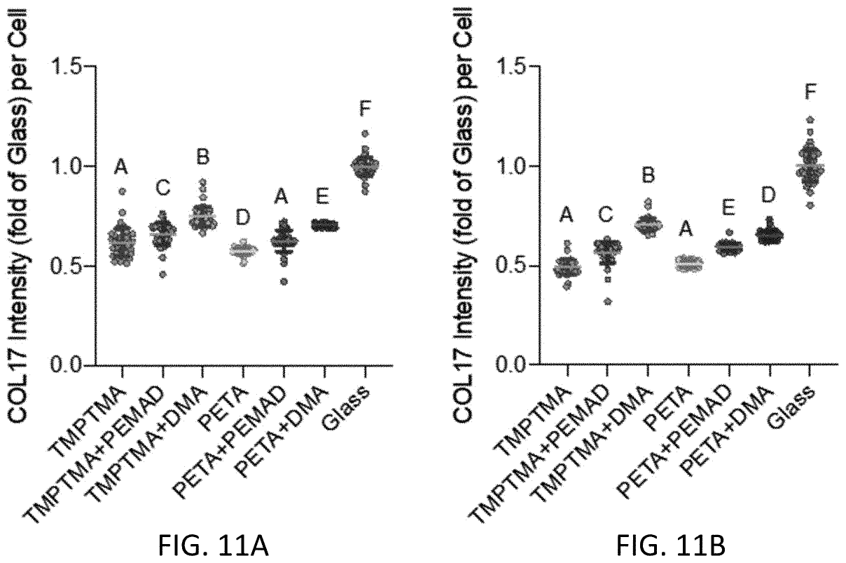

FIGS. 11A and 11B show human oral keratinocyte hemidesmosome formation (COL17; Collagen XVII) immunofluorescence intensity after 1 and 3 days of culture. Glass slides were tested as a control group for the experiment. Statistical analysis: One-way ANOVA; Tukey post-hoc; letters with different symbols were significantly different (p<0.05). Inclusion of DMA or PEMAD into TMPTMA or PETA increased immunofluorescent intensity for collagen XVII (1 and 3 days). TMPTMA-based example compositions tended higher than PETA-based example compositions. All example composition values were lower than that of the glass positive control.

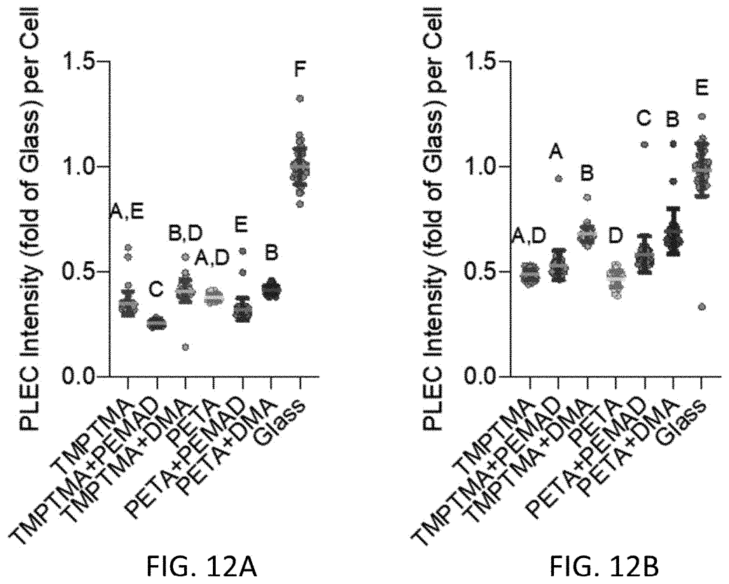

FIGS. 12A and 12B show human oral keratinocyte hemidesmosome formation (PLEC; plectin) immunofluorescence intensity after 1 and 3 days of culture, respectively. Glass slides were tested as a control group for the experiment. Statistical analysis: One-way ANOVA; Tukey post-hoc; letters with different symbols were significantly different (p<0.05). Inclusion of DMA or PEMAD into TMPTMA or PETA increased immunofluorescent intensity for plectin (1 and 3 days). TMPTMA-based example compositions tended higher than PETA-based example compositions. All example composition values were lower than that of the glass positive control. Thus, compositions described enable hemidesmosome formation compared to existing materials, which are typically cytotoxic and lack a specific biological instructive/stimulating factor

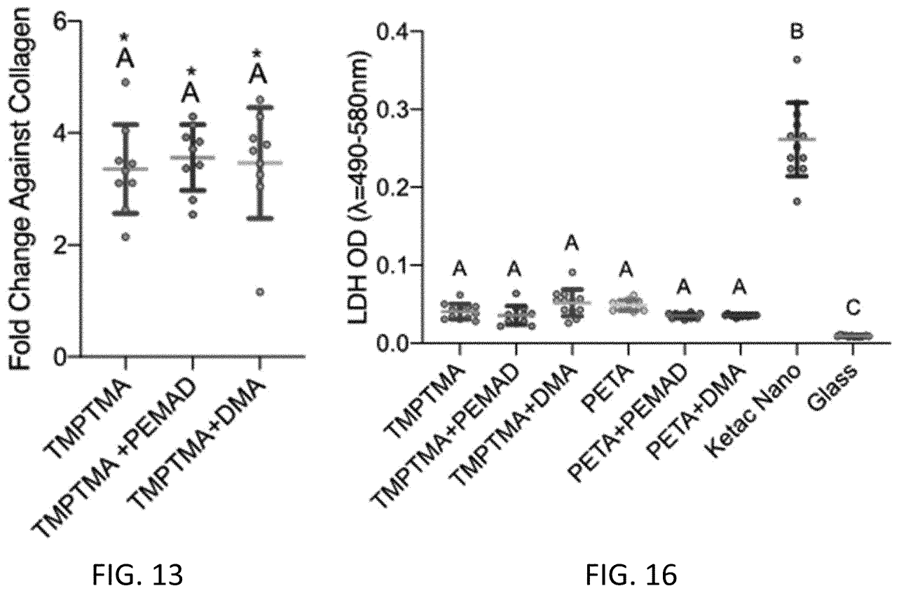

Example 7. All TMPTMA-based example compositions increase proliferation (metabolic activity) of rat-tail derived keratinocytes compared to collagen I coated glass. Primary keratinocytes were isolated from extraneous Sprague Dawley rat tails as described by others. See Li et al., Isolation and Culture of Primary Mouse Keratinocytes from Neonatal and Adult Mouse Skin. J. Vis. Exp. 2017, No. 125 DOI: 10.3791/56027. Tail skin was rinsed in ice-cold PBS and then placed in dispase digestion buffer (4 mg/mL Dispase II; DISP1, ZenBio) in defined keratinocyte serum-free media and incubated at 4° C. overnight. Skins were then washed in PBS and placed in accutase (CnT-ACCUTASE-100, ZenBio) to delaminate the dermis. The epidermis was then incubated at room temperature in fresh accutase for 20 min. Finally, the epidermis was gently agitated to begin release of keratinocytes and then placed onto the middle of each sample with fresh growth media. Samples were prepared by dipcoating example compositions onto half of a glass-slide (2975-223, Corning) that was coated following the manufacturer's recommendations with Type I collagen (5005, Advanced BioMatrix) on the other half side as an intra-tissue sample positive control for keratinocytes outgrowth. Samples were scribe cut in half (at the example composition/collagen interface) for analysis. CCK8 metabolic activity was performed as previously described except the incubation period was four hours. FIG. 13 shows keratinocyte (derived from rat tail epidermis) proliferation (CCK8 metabolic activity) of example compositions compared to collagen I coated glass slides; expressed as fold change of collagen coated surfaces. Statistical analysis: One-way ANOVA; Tukey post-hoc; letters with different symbols were significantly different (p<0.05). * indicates statistically significant different from collagen control (i.e., fold change of 1.0) for each example composition. All tested example compositions significantly increased rat-tailed derived keratinocyte proliferation compared to collagen I, a control known to increase cell proliferation. This also shows the ability of the example compositions to yield desired biological activity beyond one cell line.

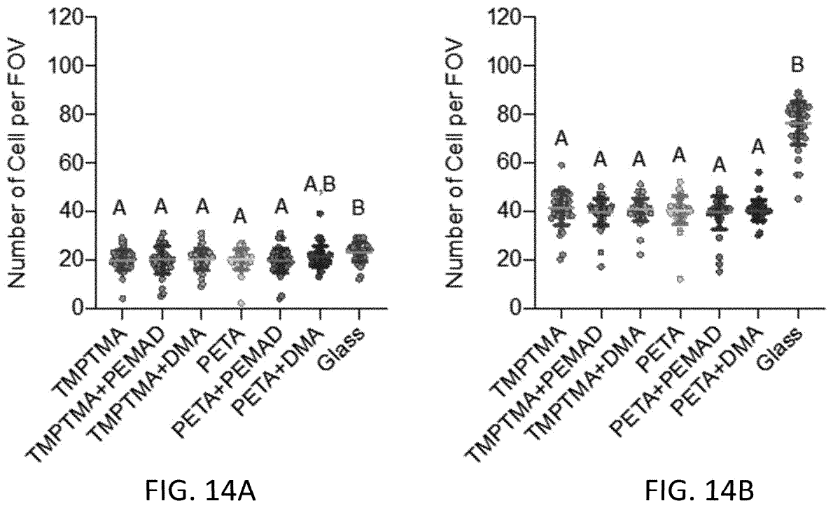

Example 8. Incorporation of DMA and PEMAD into both TMPTMA and PETA example compositions does not cause differences in fibroblast proliferation as measured by metabolic activity and number of cells. Primary human gingival fibroblasts (PCS-201-018, ATCC) were cultured in low-serum media (PCS-201-041) with 1% penicillin/streptomycin and were used between passages 2-14. The number of cells per field of view was determined as described for keratinocytes. FIGS. 14A and 14B show human gingival fibroblast proliferation (number of cells per field of view (FOV)) after 1 and 3 days of culture, respectively. Glass slides were tested as a control group for the experiment. Statistical analysis: One-way ANOVA; Tukey post-hoc; letters with different symbols were significantly different (p<0.05). Results: Inclusion of DMA or PEMAD into TMPTMA or PETA did not affect fibroblast proliferation (number of cells per field of view; 1 and 3 days). All example composition values were lower than that of the glass positive control at 3 days.

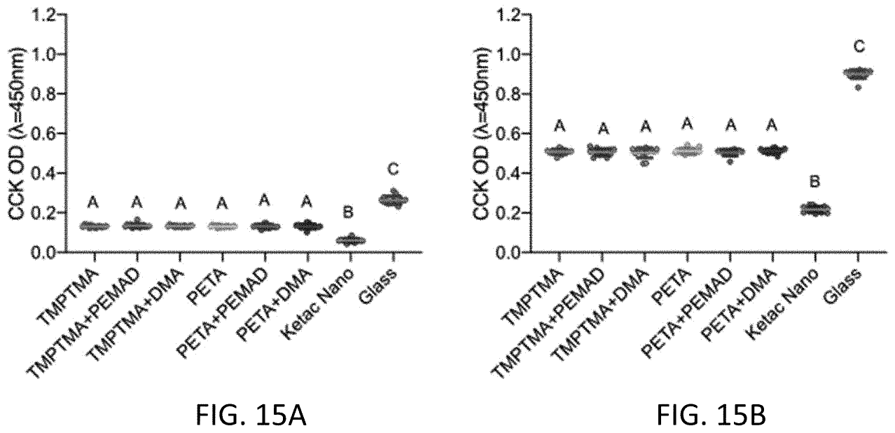

FIGS. 15A and 15B show human gingival fibroblast proliferation (CCK8 metabolic activity) after 1 and 3 days of culture, respectively. Glass slides and a commercially available restorative material (Ketac Nano) were tested as a control group for the experiment. Statistical analysis: One-way ANOVA; Tukey post-hoc; letters with different symbols were significantly different (p<0.05). Inclusion of DMA or PEMAD into TMPTMA or PETA did not affect fibroblast proliferation (metabolic activity; 1 and 3 days). All example composition values were lower than that of the glass positive control at 1 and 3 days. Ketac Nano displayed lower values than all other groups. Results in FIGS. 14A-14B and 15A-15B in combination with results in FIGS. 7 and 8 confirmed the cell proliferative effects of the materials in keratinocytes, but not in fibroblasts.

Initial keratinocyte viability was determined through lactate dehydrogenase (LDH) release. Cells were seeded as previously described and allowed to adhere for four hours. Disks were then transferred to a new wellplate. A CyQUANT colorimetric assay (C20300, Thermo Fisher) was used twenty hours later to quantify the amount of LDH in solution, per the manufacturer's instructions. FIG. 16 shows human oral keratinocyte cytotoxicity (via LDH release) after 1 day of culture. Glass slides and a commercially available restorative material (Ketac Nano) were tested as a control group for the experiment. Statistical analysis: One-way ANOVA; Tukey post-hoc; letters with different symbols were significantly different (p<0.05). Inclusion of DMA or PEMAD into TMPTMA or PETA did not affect keratinocyte viability. All example compositions values were higher than the glass control but lower than the cytotoxic Ketac Nano control. This suggests that cellular response differences seen in keratinocytes are not due to differences in material cytotoxicity.

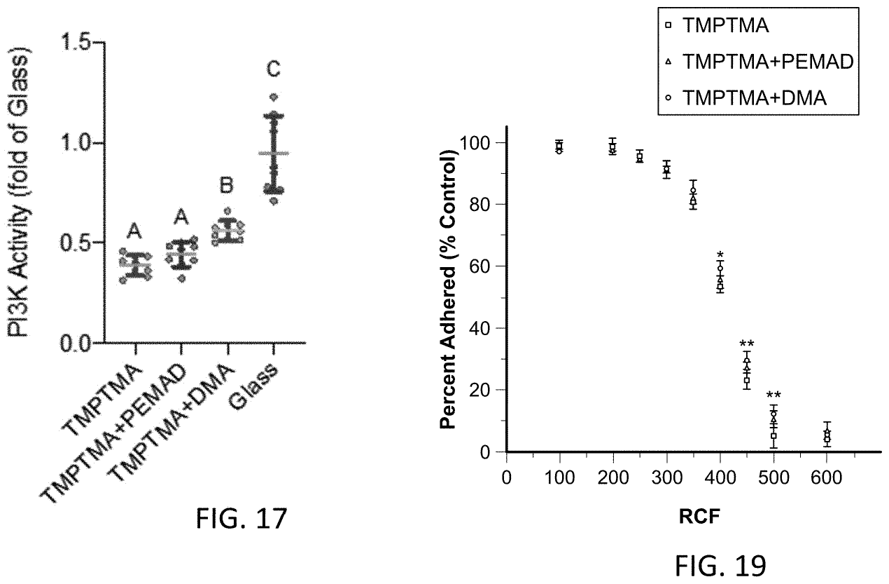

Example 9. Incorporation of DMA into TMPTMA increases PI3K (phosphoinositide 3-kinase) activity, a downstream marker of hemidesmosome formation compared to TMPTMA. PI3K activity was measured after keratinocytes were seeded and cultured as described. After 24 hours, cells were lysed (9803S, Cell Signaling Technology with (1% v/v protease inhibitor (78429, Thermo Scientific)) and lysates centrifuged (s). PI3K activity was detected with a commerically available kit (17-493, Millipore Sigma). Values were normalized to total protein content of the lystate, then to an internal positive PI3K control, and then to glass. FIG. 17 shows human oral keratinocyte PI3K (phosphoinositide 3-kinase) activity (downstream of hemidesmosome formation) via a modified ELISA. Glass slides were tested as a control group for the experiment. Statistical analysis: One-way ANOVA; Tukey post-hoc; letters with different symbols were significantly different (p<0.05). Inclusion of DMA increased PI3K, a downstream marker of mature hemidesmosome formation. Inclusion of PEMAD increased PI3K activity but it was not statistically significant.

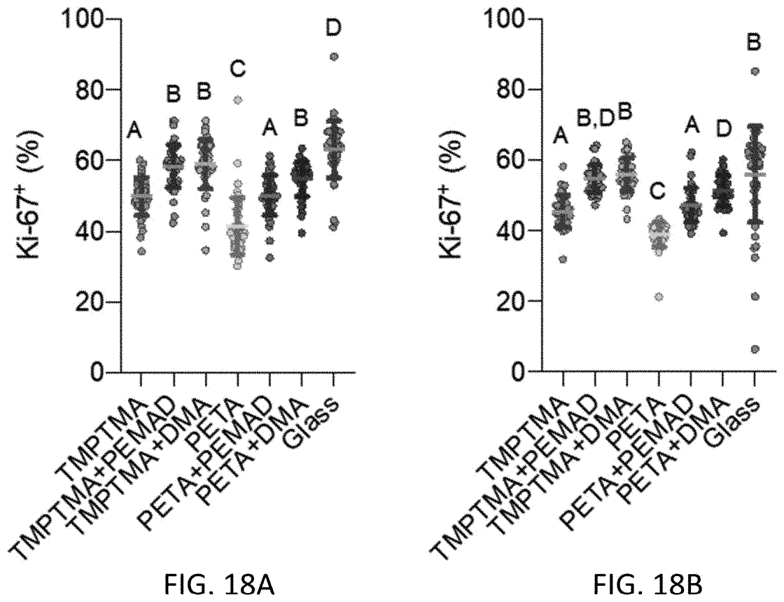

Keratinocytes were immunofluorescently stained for both Ki-67 and rhodamine-conjugated phalloidin and visualized. The percentage of cells positive for Ki-67 (rhodamine is a pan-cell marker) was determined in ImageJ. FIGS. 18A and 18B show human oral keratinocyte formation proliferation marker (Ki-67; percent of total cells positive for the marker) immunofluorescence expression after 1 and 3 days of culture, respectively. Glass slides were tested as a control group for the experiment. Statistical analysis: One-way ANOVA; Tukey post-hoc; letters with different symbols were significantly different (p<0.05). Inclusion of DMA or PEMAD into TMPTMA or PETA increased the number of cells Ki-67 positive cells (1 and 3 days). TMPTMA-based example compositions tended higher than PETA-based example compositions. All example composition values were lower than that of the glass positive control at 1 day. This further confirms the multiple lines of evidence that the inclusion of DMA or PEMAD increased keratinocyte activity.

Example 10. Incorporation of DMA and PEMAD into TMPTMA increases the mechanical adhesion of keratinocytes compared to TMPTMA. Keratinocytes were seeded as described and cultured for 2 days to quantitatively measure keratinocyte adhesion to example compositions. See Reyes, et al., A Centrifugation Cell Adhesion Assay for High-Throughput Screening of Biomaterial Surfaces. J. Biomed. Mater. Res. A 2003, 67 (1), 328-333 DOI: 10.1002/jbm.a.10122. Example compositions were placed vertically in custom, 3D-printed holders (printed with Dental SG Resin, Formlabs) in a wellplate and centrifuged at 100-500 g in culture media. The number of cells was determined by phalloidin staining as previously described before and after centrifugation (separate samples) and expressed as a percentage of cells remaining after centrifugation. FIG. 19 shows human oral keratinocyte mechanical adhesion (percent adhered compared to non-centrifuged) to example compositions after centrifugation at varying RCF (g) values. Statistical analysis: * indicates a statistically significant difference compared to other groups. Inclusion of DMA or PEMAD into TMPTMA increased mechanical adhesion of the cells to the substrate, suggesting the functionality of increased hemidesmosome formation.

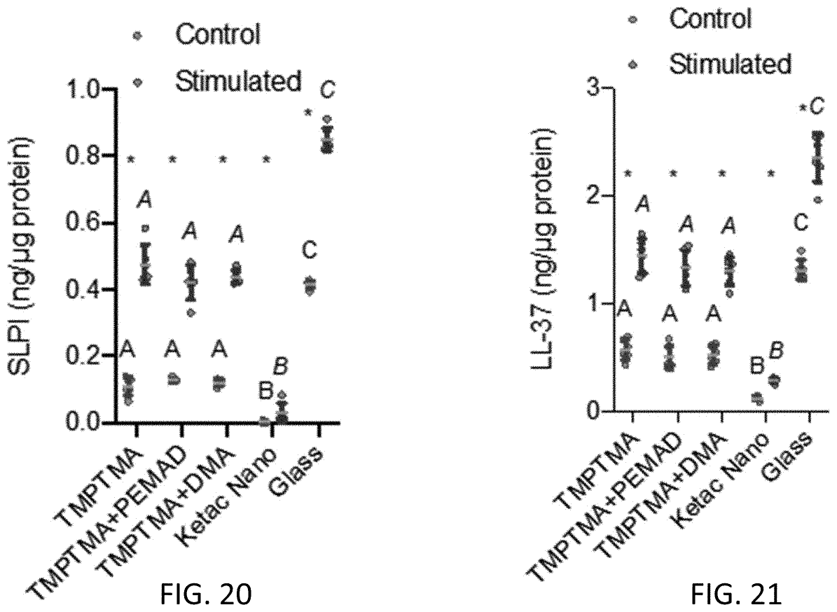

Example 11. Incorporation of DMA and PEMAD into TMPTMA does not alter SLPI (secretory leukocyte peptidase inhibitor), including in response to interleukin 1α stimulation, and is expressed at a higher level than an existing restorative material. Keratinocytes were seeded as described and cultured for 24 hours on example compositions. After this, Interleukin 1 alpha (IL-1α; 200-LA, R&D systems) was used to stimulate (10 ng/mL for 24 hours) SLPI production. See Bando, et al., Interleukin-1α Regulates Antimicrobial Peptide Expression in Human Keratinocytes. Immunol. Cell Biol. 2007, 85 (7), 532-537 DOI: 10.1038/sj.icb.7100078. Cells were lysed as previously. SLPI protein context was detected with an ELISA per the manufacturer's instructions. Total protein was used for normalization. FIG. 20 shows human oral keratinocyte SLPI (secretory leukocyte peptidase inhibitor) expression at homeostasis (control) and after exposure to IL-1α (interleukin 1α; stimulated). A commercially available restorative material (Ketac Nano) and glass slides were tested as a control group for the experiment. Statistical analysis: One-way ANOVA; Tukey post-hoc; letters with different symbols were significantly different (p<0.05). Control groups (such as A) and stimulated groups (such as italicized groups; A) were compared separately. * indicates statistically significant difference from control, non-stimulated for each group. Inclusion of DMA or PEMAD into TMPTMA does not affect keratinocytes SLPI production, a critical protein marker for soft tissue function, compared to TMPTMA. SLPI expression is responsive to stimulation and higher than Ketac Nano, but lower than the positive control glass.

Example 12. Incorporation of DMA and PEMAD into TMPTA does not after LL-37, including in response to LPS (lipopolysaccharide) stimulation, and is expressed at a higher level than an existing restorative material. Keratinocytes were seeded as described and cultured for 24 hours on example compositions. After this, lipopolysaccharide (LPS; derived from P. gingivalis; tlrl-pglps, Invivogen) was used to stimulate (100 ng/mL for 12 hours) LL-37 production. See Nell et al., Bacterial Products Increase Expression of the Human Cathelicidin HCAP-18/LL-37 in Cultured Human Sinus Epithelial Cells. FEMS Immunol. Med. Microbiol. 2004, 42 (2), 225-231 DOI: 10.1016/j.femsim.2004.05.013. See also Kim et al, Expression and Modulation of LL-37 in Normal Human Keratinocytes, HaCaT Cells, and Inflammatory Skin Diseases. J. Korean Med. Sci. 2005, 20 (4), 649 DOI: 10.3346/jkms.2005.20.4.649. Cells were lysed (150 mM NaCl, 50 mM Tris HCl, 1% Triton X-100 and 1% v/v protease inhibitor (78429, Thermo Scientific)) and lysates centrifuged (12,000 g, 10 min). LL-37 protein content was detected with an ELISA per the manufacturer's instructions. Total protein was used for normalization. FIG. 21 shows human oral keratinocyte LL-37 expression at homeostasis and after exposure to LPS (lipopolysaccharide; stimulated). A commercially available restorative material (Ketac Nano) and glass slides were tested as a control group for the experiment. Statistical analysis: One-way ANOVA; Tukey post-hoc; letters with different symbols were significantly different (p<0.05). Control groups (such as A) and stimulated groups (such as italicized groups; A) were compared separately. * indicates statistically significant difference from control, non-stimulated for each group. Inclusion of DMA or PEMAD into TMPTMA does not affect keratinocytes LL-37 production, a critical protein marker for soft tissue function, compared to TMPTMA. SLPI expression is responsive to stimulation and higher than Ketac Nano, but lower than the positive control glass.

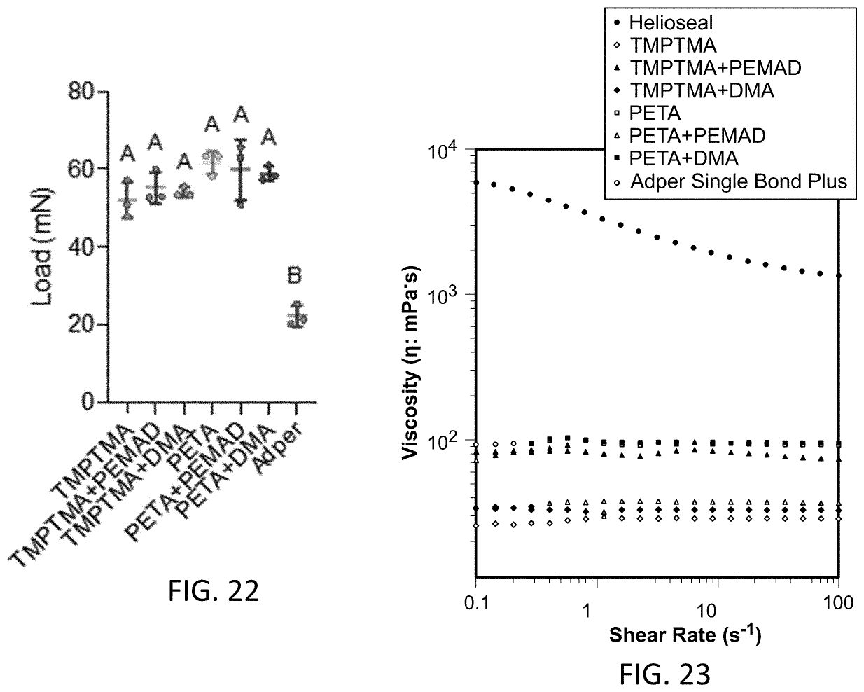

Example 13. Coatings of all example compositions on a dental restorative material yields higher failure loads during a scratch test than a commercially available dental adhesive. Ketac Nano disks were polished (SiC; 200 and 600) and then example compositions were spin coated (Laurell WS-650) at 1500 RPM for 60 s with a ramp rate of 750 RPM per s. SEM crossections were used to measure film thickness, which was nominally similar across example compositions; ca. 30 μm. Scratch testing (TI980 TriboIndentor, Bruker; diamond tip (TI-0092) with a nominal conical radius of 5 μm) was performed in peak displacement mode with 0-18 μm beginning to end displacement and a maximum lateral displacement of 100 μm. The first well-defined failure event was determined from lateral displacement vs. normal force plots. FIG. 22 shows failure load during scratch test for mechanical attachment to substrate after a thin layer of resin example composition is placed on a commercial Class V filling material (Z250, 3M Oral Care Solutions). A commercial dental adhesive (Adper SingleBond Plus, 3M Oral Care Solutions) was tested as a control. Statistical analysis: One-way ANOVA; Tukey post-hoc; letters with different symbols were significantly different (p<0.05). No differences were seen in adherence to a commercially available restorative material between any of the example compositions. Loads of the example compositions to failure were markedly higher than an existing dental adhesive, Adper.

Example 14. Addition of DMA and PEMAD into TMPTMA and PETA example compositions does not alter their viscosity, all of which are similar to a commercially available dental adhesive. Parallel plate rheometry (∅=25 mm; 1 mm gap) was performed to compare viscosities of example compositions on an MCR 302 (Anton Paar). A frequency sweep test was performed from 100 rad/s to 0.01 rad/s after pre-shearing. FIG. 23 shows rheology shear rate sweep of example compositions. A commercial dental adhesive (Adper SingleBond Plus) and a commercial pit and fissure sealant (Helioseal) were tested as a controls. Statistical analysis: One-way ANOVA; Tukey post-hoc; letters with different symbols were significantly different (p<0.05). PETA-based example compositions were more viscous than TMPTMA. The inclusion of DMA or PEMAD had trivial effects. Viscosities of example compositions were similar to the existing commercially available materials Adper and less viscous than the shear-thinning Helioseal.

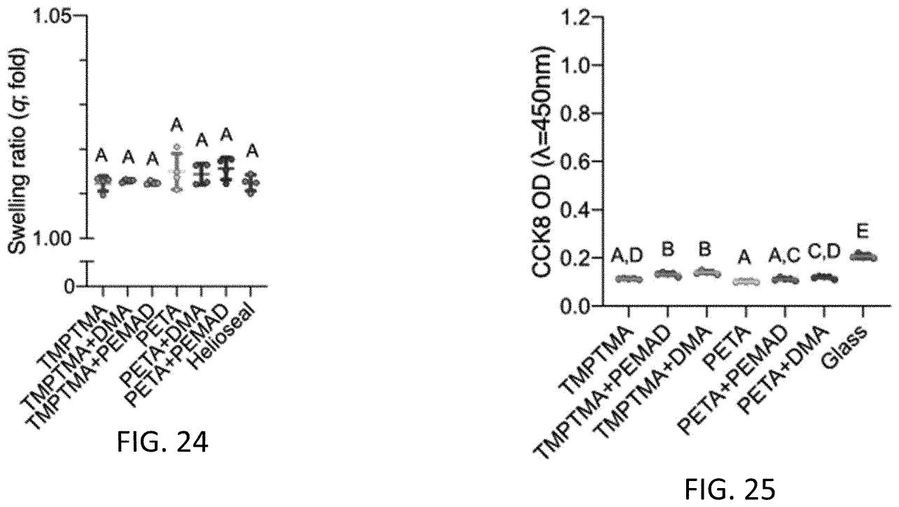

Example 15. Incorporation of DMA and PEMAD into both TMPTMA and PETA example compositions does not affect the swelling ratio of the resultant example compositions. The swelling ratio (q; based on mass) was determined based on mass before and after example composition in incubation TBS (pH=8.5, 37° C.) for 24 hours. FIG. 24 shows swelling ratio (comparison of weight before and after immersion in phosphate buffered saline) of example compositions. A commercially available restorative material (Helioseal) was used as a control. Statistical analysis: One-way ANOVA; Tukey post-hoc; letters with different symbols were significantly different (p<0.05). All example compositions displayed similar swelling (q) as Helioseal.

Example 16. All TMPTMA and PETA example compositions retain their activity of increased keratinocyte proliferation following one week of water-mediated degradation. Example compositions were immersed in TBS (pH=8.5, equivalent for gingival crevicular fluid) for one week at 37° C. After this, samples were dessicated for 24 hours and then metabolic activity was determined with CCK8 at 1 day of culture, as previously described. FIG. 25 shows human oral keratinocyte proliferation (metabolic activity; 1 day) on TMPTMA and PETA example compositions after hydrolytic degradation for 1 week. Glass slides were tested as a control group for the experiment. Statistical analysis: One-way ANOVA; Tukey post-hoc; letters with different symbols were significantly different (p<0.05). The increased proliferation (metabolic activity) due to inclusion of DMA or PEMAD was retained in both TMPTMA and PETA groups following aging in water. The positive glass control was higher than all other groups.

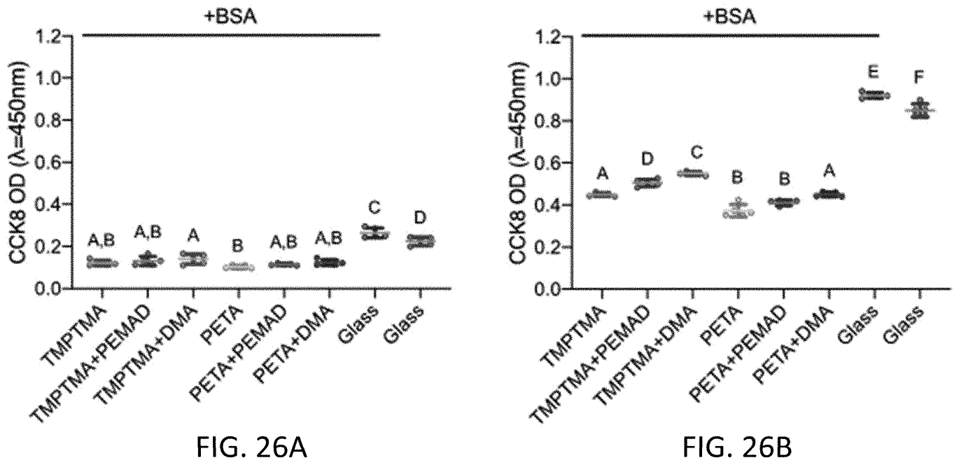

Example 17. Fouling of all example compositions with bovine serum albumin does not hinder resultant increased proliferation (metabolic activity and number of cells) and hemidesmosome upregulation (collagen XVII) from incorporation of DMA and PEMAD into both TMPTMA and PETA example compositions. CCK8 proliferation was determined as described after example compositions were immersed in 5% bovine serum albumin (BSA) in PBS for 3.5 hours at room temperature. Example compositions were washed in PBS thrice before cell culture. FIGS. 26A and 26B show human oral keratinocytes proliferation (CCK8 metabolic activity) after 1 and 3 days, respectively, of culture after fouling of example compositions with bovine serum albumin (BSA). Glass slides (with and without BSA) were tested as a control group for the experiment. Statistical analysis: One-way ANOVA; Tukey post-hoc; letters with different symbols were significantly different (p<0.05). The increased proliferation (metabolic activity) due to inclusion of DMA or PEMAD was retained in both TMPTMA and PETA groups following BSA fouling at 3 days of culture. The positive BSA-fouled glass control was higher than all other groups, including virgin glass.

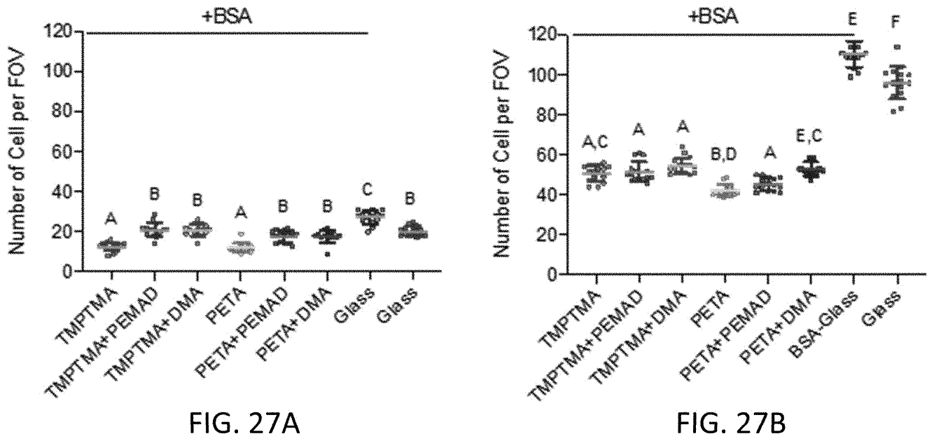

Proliferation (number of cells per field of view) was determined as described after example compositions were immersed in 5% BSA in PBS for 3.5 hours at room temperature. Example compositions were washed in PBS thrice before cell culture. FIGS. 27A and 27B show human oral keratinocytes proliferation (number of cells per field of view (FOV)) after 1 and 3 days, respectively, of culture after fouling of example compositions with BSA. Glass slides (with and without BSA) were tested as a control group for the experiment. Statistical analysis: One-way ANOVA; Tukey post-hoc; letters with different symbols were significantly different (p<0.05). The increased proliferation (number of cells) due to inclusion of DMA or PEMAD was generally retained in both TMPTMA and PETA groups following BSA fouling at 1 and 3 days. The positive BSA-fouled glass control was higher than all other groups, including virgin glass.

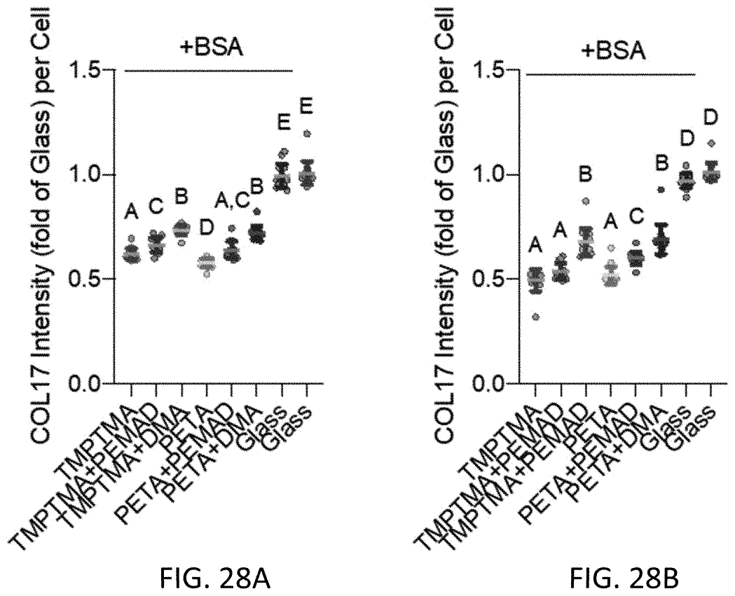

Collagen XVII immunofluorescent staining was performed as previously described. FIGS. 28A and 28B show human oral keratinocyte hemidesmosome formation (Collagen XVII) immunofluorescence intensity after 1 and 3 days, respectively, of culture after fouling of example compositions with BSA. Glass slides (with and without BSA) were tested as a control group for the experiment. Statistical analysis: One-way ANOVA; Tukey post-hoc; letters with different symbols were significantly different (p<0.05). The increased hemidesmosome formation due to inclusion of DMA or PEMAD was generally retained in both TMPTMA and PETA groups following BSA fouling at 1 and 3 days. Glass controls were higher than other groups.

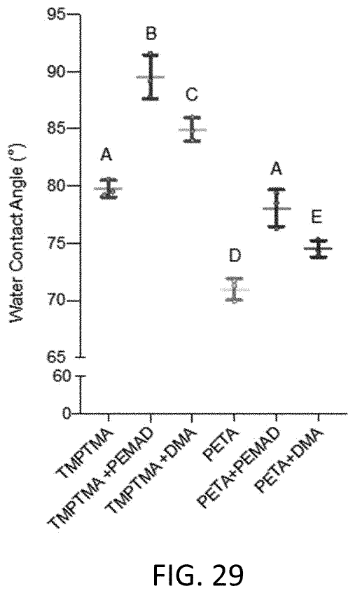

Example 18. Incorporation of DMA and PEMAD into both TMPTMA and PETA example compositions alters wettability of the example compositions. Water contact angle was measured at equilibrium with a sessile-drop method (2.0 μL deionized water) with a contact angle meter (DM-CE1, Kyowa, Japan). FIG. 29 shows sessile drop water contact angle of example compositions and representative images, respectively. Statistical analysis: One-way ANOVA; Tukey post-hoc; letters with different symbols were significantly different (p<0.05). Inclusion of DMA increased the water contact angle of TMPTMA and PETA by approximately 5° whereas the inclusion of PEMAD increased the water contact angle by approximately 10.° PETA-based example compositions displayed lower contact angles than TMPTMA.

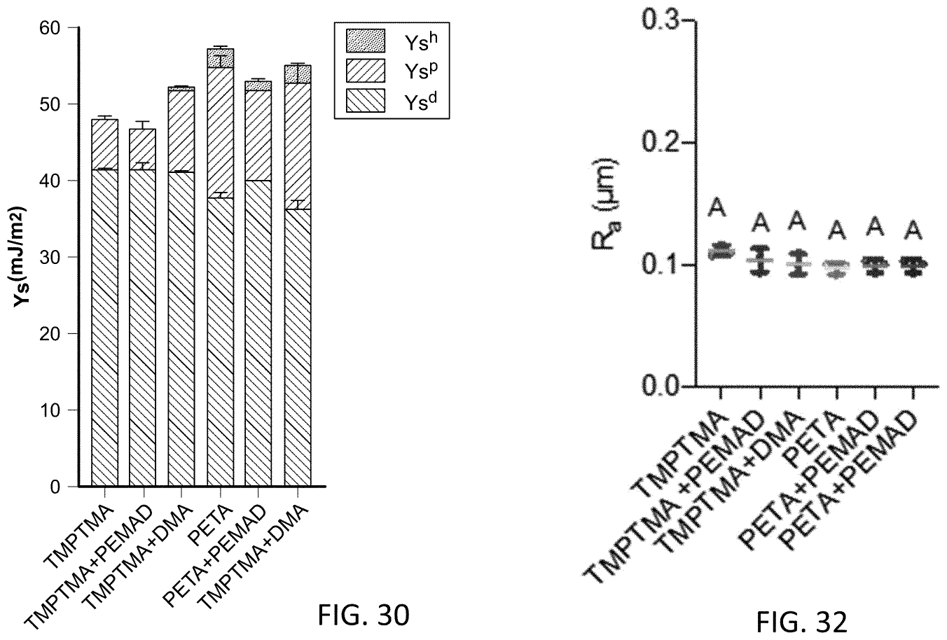

Example 19. Incorporation of DMA and PEMAD into both TMPTMA and PETA example compositions alters the resultant surface free energy. Surface free energy (SFE) was determined using a three-probe approach with deionized water, 1-bromonaphtene, and diiodomethane as previously described. See Tsujimoto, et al., Enamel Bonding of Single-Step Self-Etch Adhesives: Influence of Surface Energy Characteristics. J. Dent. 2010, 38 (2), 123-130 DOI: 10.1016/j.jdent.2009.09.011. Values are reported as γ^{d}, γ^{p}, and γ^{h} components of SFE arising from the dispersion force, the polar (permanent and induced) force, and the hydrogen-bonding force, respectively. See Hata et al., Estimation of the Surface Energy of Polymer Solids. J. Adhes. 1987, 21 (3-4), 177-194 DOI: 10.1080/00218468708074968. FIG. 30 shows surface free energy (SFE; γ^{s}) of example compositions determined with contact angles. Values are reported as γ^{d}, γ^{p}, and γ^{h} components of SFE arising from the dispersion force, the polar (permanent and induced) force, and the hydrogen-bonding force, respectively. TMPTMA-based example compositions showed a higher γ^{p} than TMPTMA.

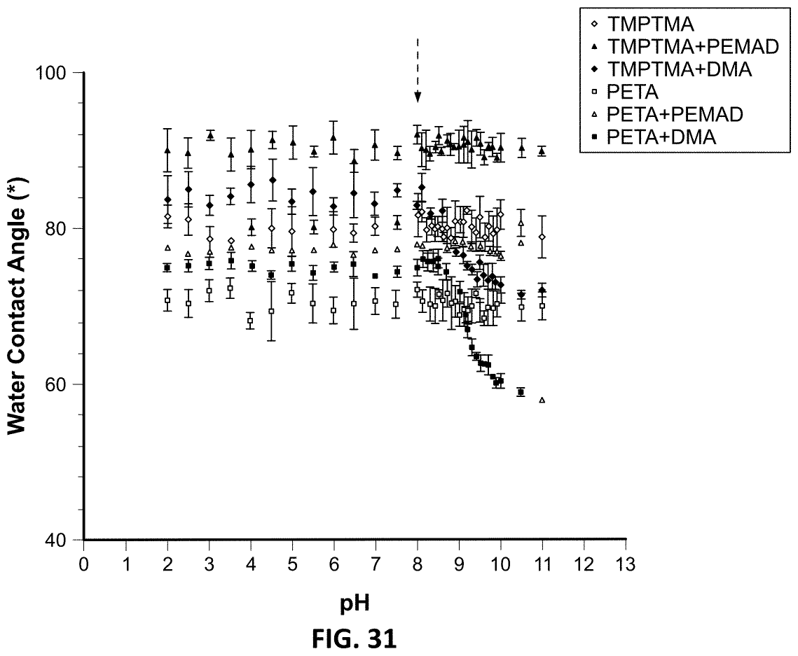

Example 20. Incorporation of DMA, but not PEMAD, into both TMPTMA and PETA example compositions imbues the resultant example composition with a surface charge. The point of zero charge (PZC) of example compositions was determined using a contact angle-based approach. See Horiuchi, et al., Calculation of the Surface Potential and Surface Charge Density by Measurement of the Three-Phase Contact Angle. J. Colloid Interface Sci. 2012, 385 (1), 218-224 DOI: 10.1016/j.jcis.2012.06.078. Electrostatic interactions between the solid and liquid phase are minimized and lead to the maximum contact angle at the PZC. See Hanly et al., Electrostatics and Metal Oxide Wettability. J. Phys. Chem. C 2011, 115 (30), 14914-14921 DOI: 10.1021/jp203714a. Solutions with varying pHs were prepared with hydrochloric acid and alkali solutions with varying pHs were prepared with sodium hydroxide for a range (pH=2-12; increments of 0.5) of solutions with equivalent ionic strengths (c=0.05 mol/L; determined with Buffer Maker ChemBuddy). Contact angle was measured as described previously but in at least 90% relative humidity (TAYLOR digital hygrometer). FIG. 31 shows angle of various solutions of water with similar ionic strengths at pH=2 to pH=12 on all example compositions. A decrease in contact angle in response to varying of the solution pH suggests presence of a surface charge, as denoted by the arrow for groups TMPTMA+DMA and PETA+PEMAD at around pH=8.0. The reduction in contact angle for example compositions with DMA at around pH=8.0 (and higher pHs) indicates the presence of a surface charge.

Example 21. Incorporation of DMA and PEMAD into both TMPTMA and PETA example compositions does not affect surface roughness (Rₐ). A white-light confocal laser microscope (HS200, Hyphenated Systems) was used to measure surface roughness (Rₐ; arithmetic mean of height deviations) of example compositions. Example compositions were scanned, filtered with a Gaussian waviness filter (λ_{c}=20), and smoothened using a Circular Hann window smoothing (0.70 μm diameter). FIG. 32 shows surface roughness (Rₐ) of example compositions determined with a white-light confocal laser microscope. Statistical analysis: One-way ANOVA; Tukey post-hoc; letters with different symbols were significantly different (p<0.05). No differences were seen in Ra between any of the groups.

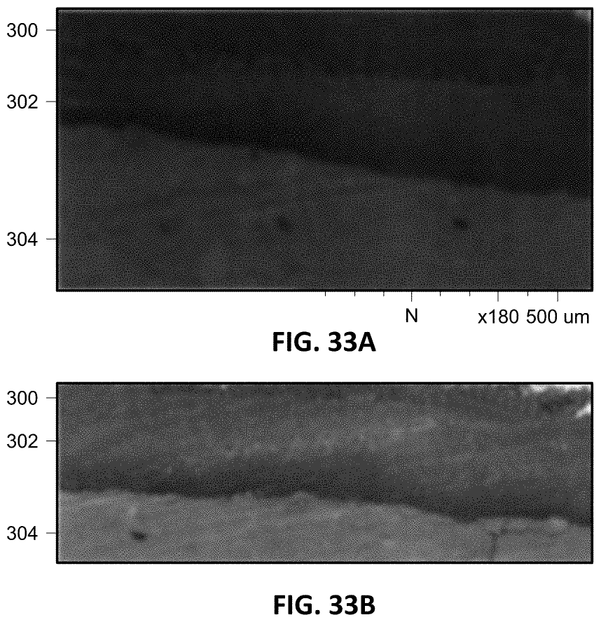

Example 22. A TMPTMA example composition, after simulated toothbrush equivalent to 9.25 years, is retained after coating on a representative restorative materials surface. Resistance to delamination of the TMPTA example composition was testing using a toothbrush machine following the general principles of ISO Standard 14569-1 (2007). The TMPTA example composition was applied to Ketac Nano with a dental micro applicator brush and photopolyermized. This specimen was then mounted in dental impression material for 92,499 cycles of toothbrushing of a 2.1N load at 2 Hz in a 2:1 (vol) water to toothpaste (Crest Regular Paste) ratio in the MDRCBB toothbrusher (Dr. Best Flex Plus toothbrush). See Ko, et al. Assay Development for Toothbrush Damage on Hard Tissues. J. Dent. Res. 1996, 75, 1413-1413. This corresponds to approximately 9.25 years of brushing. See Teixeira, et al., In Vitro Toothbrush-Dentifrice Abrasion of Two Restorative Composites. J. Esthet. Restor. Dent. 2005, 17 (3), 172-180. Afterwards, the specimen was embedded, sectioned, and examined with scanning electron microscopy. FIG. 33A shows a scanning electron micrograph of a cross section with embed resin 300 and example composition (TMPTMA) 302 adhered to KN 304 before tooth brushing. FIG. 33B shows a scanning electron micrograph of cross section with embed resin 300 and example composition 302 similarly adhered to KN 304 after tooth brushing. Thus, TMPTMA, when applied as a thin film on a dental restorative material, does not delaminate following simulated toothbrushing for an extended timeframe.

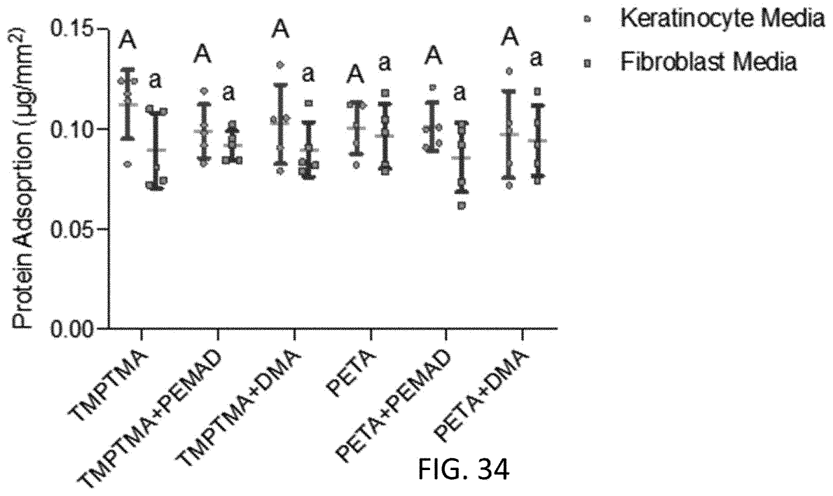

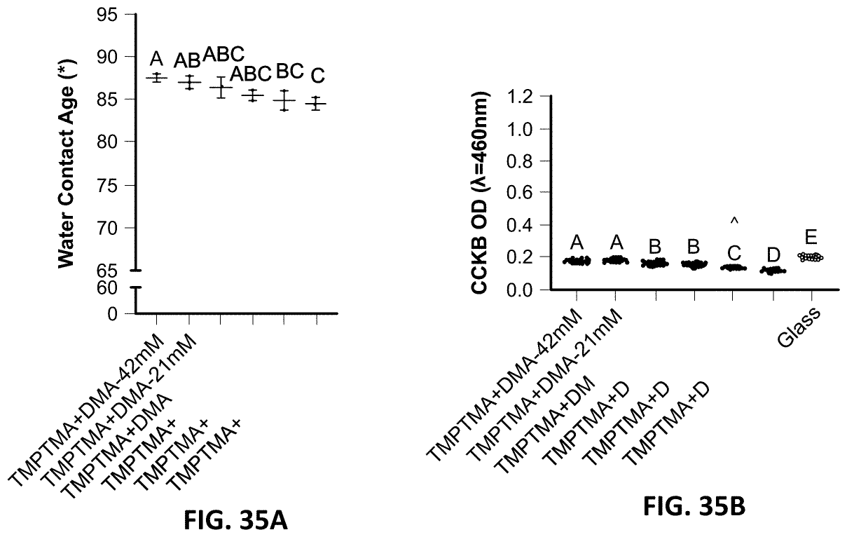

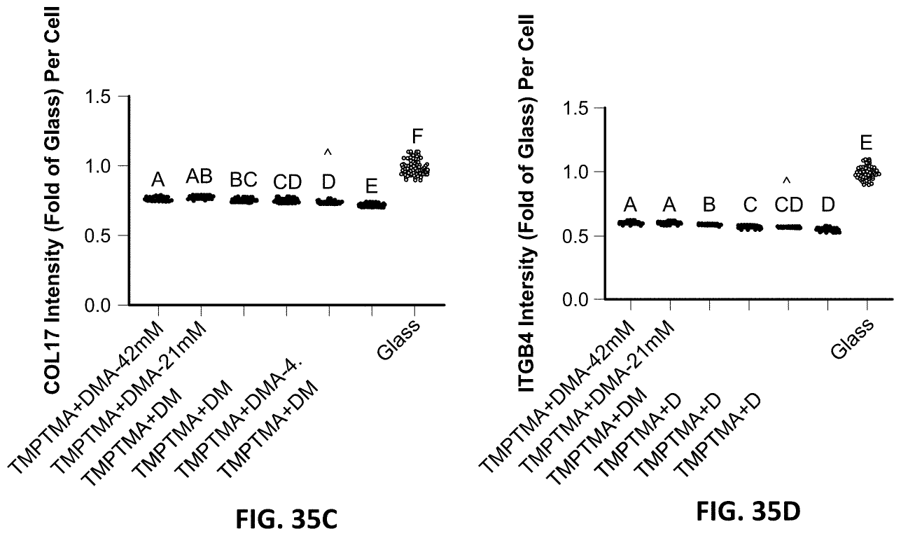

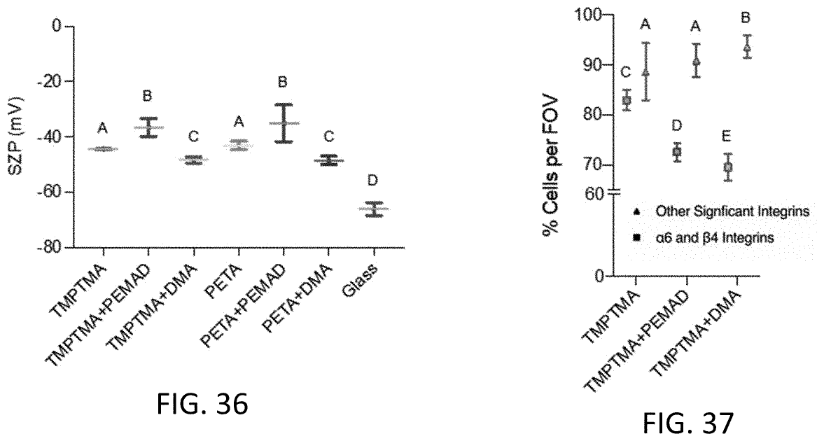

Example 23. Incorporation of DMA and PEMAD into both TMPTMA and PETA example compositions does not affect the amount of protein adsorbed after immersion in either keratinocyte or fibroblast medium. Serum proteins adsorbed on example compositions were measured with a micro bicinchoninic acid (BCA; 23235, Thermo Fisher). Samples were equilibrated in PBS for two hours and then incubated (37° C.) for 24 hours in either keratinocyte or fibroblast media. After this, disks were rinsed in PBS five times and then desorbed in 2% (v/v) Triton X-100 in PBS for 60 minutes total with 20 minutes of ultrasonnication. Protein concentration was then determined using a commerically available kit with a standard curve and normalized to the nominal disk surface area. FIG. 34 shows protein adsorption (via BCA assay) from keratinocytes and fibroblast media on example compositions. Statistical analysis: One-way ANOVA; Tukey post-hoc; letters with different symbols were significantly different (p<0.05). Each media was separately compared (i.e., upper case letters for keratinocyte media vs. lower case letters for fibroblast media). No differences were seen between any of the example compositions in terms of protein adsorption from either media. This suggests that differences in biological performance are not due to protein adsorption.

Example 24. Incorporation of DMA into TMPTMA example composition yields a concentration-dependent change in water contact angle, keratinocyte metabolic activity, and keratinocyte hemidesmosome formation (Collagen XVII and integrin (34). DMA was dissolved in a blend of 90% ethyl ether and 10% deionized water (final concentration ranging from 2.1-42 mM) and used to prepare TMTMA+DMA as described. Statistical analysis: One-way ANOVA; Tukey post-hoc; letters with different symbols were significantly different (p<0.05). The \hat{ } denotes the concentration (4.2 mM) used for all other experiments presented. Concentration-dependent changes in water contact angle lead to concomitant concentration-dependent changes in keratinocyte responses, namely hemidesmosome formation (Collagen XVII and integrin β4).

Example 25. Incorporation of DMA and PEMAD into both TMPTMA and PETA example compositions alters surface zeta potential (SZP; mV) of the example compositions. Surface zeta potential (SZP) was measured by first placing each composition and control glass in a ZEN1020 (Malvern Panalytical) potential cell. Tracer particles (ZTS1240, Malvern Panalytical) were diluted in phosphate buffered saline. Diluted tracer particles were added to a cuvette with the ZEN1020 and then inserted into the Zetasizer Nano-Z S90 (Malvern Panalytical). Statistical analysis: One-way ANOVA; Tukey post-hoc; letters with different symbols were significantly different (p<0.05). Inclusion of PEMAD into formulations makes SZP less negative whereas inclusion of DMA makes SZP more negative. Thus, surface physicochemistry enabled by PEMAD and DMA inclusion—here, namely charge—controls hemidesmosome formation.

Example 26. Compositions for soft tissue attachment alter integrins human oral keratinocytes use to adhere, compared to IgG control, as determined by integrin antibody blocking. Hemidesmosome-related integrins α6, β4 and co-α6 and -β4 blocking (▴) were pooled and compared to the pooled mean of all other integrins with significantly reduced adhesion (▪) on TMPTMA and PETA example compositions after 20 hours of culture. Keratinocytes were incubated with blocking antibodies (ECM340 and ECM440, Merck Millipore; α1, α2, α3, α4, α5, α6, αv, β1, β2, β3, β4, β5, or α6 plus β4 integrin subunits simultaneously; azides were removed via dialysis) at 5 μg mL-l for 15 minutes. A mouse IgG antibody was used as a control (10400C, Invitrogen). Then, cells were seeded as previously described and counting was performed as described at 20 hours. For analysis, mean values that were significantly different from the IgG control (excluding α6, β4, and α6 plus β4; these are integrin subunits necessary for HD formation) were combined for comparison to the combined mean of α6, β4, and α6 plus β4 mean values. Statistical analysis: One-way ANOVA; Tukey post-hoc; letters with different symbols were significantly different (p<0.05). Formulation physicochemistry—namely inclusion of DMA or PEMAD—alters integrins used for keratinocyte adhesion to compositions for soft tissue attachment

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. An ultraviolet-photopolymerizable composition comprising:
trimethylolpropane tris(3-mercaptopropionate) (TMTMP);
a photoinitiator;
a solvent; and
one or more of trimethylolpropane trimethacrylate (TMPTMA), pentaerythritol triacrylate (PETA), dopamine methacrylamide (DMA), and n-phenethylmethacrylamide (PEMAD).

2. The composition of claim 1, wherein the photoinitiator comprises dimethylol propionic acid (DMPA).

3. The composition of claim 1, wherein the composition comprises 0.5 w/v to 5 w/v of the photoinitiator.

4. The composition of claim 3, wherein the composition comprises 0.5 w/v to 2 w/v of the photoinitiator.

5. The composition of claim 1, wherein the solvent comprises one or more of water, methanol, ethanol, acetone, tetrahydrofuran (THF), ethyl ether, and dichloromethane (DCM).

6. The composition of claim 1, wherein the composition comprises 5% v/v to 40% v/v of the solvent.

7. The composition of claim 1, wherein the composition comprises TMPTMA and PETA.

8. The composition of claim 7, wherein a molar ratio of TMPTMA and PETA to TMTMP is in a range of 1:0 to 4:0.

9. The composition of claim 7, wherein the composition comprises up to 50 mM DMA.

10. The composition of claim 7, wherein the composition comprises up to 50 mM PEMAD.

11. The composition of claim 10, wherein the composition comprises up to 50 mM DMA.

12. A method of coating a substrate with a polymeric coating, the method comprising:
contacting a substrate with the composition of claim 1;
irradiating the composition with ultraviolet radiation to yield a polymeric coating on the substrate.

13. The method of claim 12, wherein the ultraviolet radiation has a wavelength of approximately 365 nm.

14. The method of claim 12, wherein the irradiating comprises irradiation at an intensity of at least 2 mWcm⁻² for at least 20 seconds.

15. The method of claim 12, further comprising contacting the polymeric coating with soft tissue.

16. A coated substrate formed by the method of claim 12.

17. The coated substrate of claim 16, wherein the substrate comprises a device configured to be at least partially inserted in a mammalian body or in contact with soft tissue in a mammalian body.

18. The coated substrate of claim 17, wherein the soft tissue comprises skin or oral mucosa.

19. The coated substrate of claim 17, wherein the substrate comprises a dental restoration, a catheter, or an osseointegrated, percutaneous device.

20. The coated substrate of claim 19, wherein the osseointegrated percutaneous device comprises an orthopedic limb prosthesis, a dental implant, or a bone-anchored hearing aid.

21. The coated substrate of claim 19, wherein the catheter comprises a dialysis catheter or a ventricular assisted device.

22. The coated substrate of claim 19, wherein the dental restoration is at least partially in a root of a tooth.

23. A method of treating a substrate, the method comprising:
contacting the substrate with the composition of claim 1, wherein the substrate comprises a dental restoration, a catheter, or an osseointegrated percutaneous device; and
irradiating the composition with ultraviolet radiation to yield a polymeric coating on the substrate, wherein the polymeric coating promotes soft tissue attachment to the substrate.
